(19) United States
(12) Patent Application Publication
(10) Pub. No.: US 2010/0323441 A1
(43) Pub. Date: Dec. 23, 2010

(54) METHOD OF COLLECTION AND PRESERVATION OF FLUIDS AND/OR MATERIALS, IN PARTICULAR OF ORGANIC FLUIDS AND/OR MATERIALS CONTAINING STEM CELLS, AND DEVICE EMPLOYABLE IN SUCH METHOD

(75) Inventors: Federico Maggi, Colazza (IT); Marco Giovanni Reguzzoni, Busto Arsibio (IT); Giuseppe Simoni, Pavia (IT); Simone Maccagnan, Castronno (IT)

Correspondence Address: Pearne & Gordon LLP, 1801 East 9th Street, Suite 1200, Cleveland, OH, 44114-3108, US

(73) Assignee: BIOCELL CENTER S.P.A., 21052 BUSTO ARSIZIO (VARESE) (IT)

(21) Appl. No.: 12/747,437

(22) PCT Filed: Dec. 13, 2007
(86) PCT No.: PCT/IT07/00868

§ 371 Date: Sep. 10, 2010

(51) Int. Cl. C12N 5/0735; C12M 1/00

(52) U.S. Cl. 435/374; 435/307.1

(57) ABSTRACT

A method of collection and preservation of a fluid and/or a material comprises a step of determining a sampling volume that is closed and/or separated from the external environment, a step of taking a predetermined amount of fluid and/or material from the sampling volume and a step of confining the fluid and/or material into a collection volume; the step of taking the fluid and/or material and the step of confining the fluid and/or material are simultaneous with each other and contemporaneous with a step of maintaining the hydraulic and/or pneumatic and/or microbiological isolation between the sampling volume and the collection volume.

The present invention relates to a method of collection and preservation of fluids and/or materials, in particular of organic fluids and/or materials containing stem cells as well as to a device to be used in such a method; both the device and method are employable in different operating fields, such as sampling and preservation of organic fluids in the clinical/laboratorial sphere.

It is known that in different production fields sampling of a fluid substance from a given closed volume is made necessary, as it may happens in prenatal medical examples, and also as far as working of some electronic printed circuits is concerned.

In the first one of the two application fields mentioned above by way of example, while tests such as amniocentesis and CVS (i.e. Chorionic Villus Sampling) are being executed, a first step is in particular contemplated in which a first sampling of amniotic liquid (about 3 cc) is carried out by means of a suitably positioned syringe so as to perforate the pregnant woman's abdominal wall and placenta in the less invasive manner as possible; subsequently, while the needle is maintained implanted during the first sampling, the syringe body is changed in order to obtain a second more voluminous sampling (about 10 cc of the amniotic liquid).

Generally, the liquid taken during the first sampling is discarded, essentially due to the fact that insertion of the needle through the abdominal wall and placenta gives rise to creation of a substantially cylindrical residue of the mother's organic tissue (that in the particular field is usually referred to as “frustule” or “fragment”), which obviously interferes with the quality of the taken sample of fluid and/or material.

It is however to be pointed out that presently the known art does not contemplate any method involving long-term use of the amniotic liquid or chorionic villi, above all in connection with the possibility of preserving, taking out and subsequently manipulating the stem cells that can be found suspended therein.

The second application field mentioned above, on the contrary, involves a particular method of printing electronic circuits in which a particular gel is used that covers the board under working (which in turn is “mapped” by means of a laser beam or the like); in this method, the physico-chemical features of the gel adversely affect the conductivity of the electronic circuits printed by means of the laser beam (passing through the gel itself), and it is therefore essential that samples of the gel be carried out periodically in order to check composition of same.

In the application examples briefly described above (but also in many other operating fields) it is therefore necessary to operate on a given substance than can be in the fluid state and is adapted to be analysed/examined; at the same time, this substance is to be maintained in an operating volume that must be as much as possible isolated from external agents (atmosphere, non-sterile environments, foreign substances and so on).

Other possible fields in which sampling operations are to be executed can be for example found in the food industry (sampling of serum in cheese factories, or of wine under maturation in the wine-making industry, or still of various substances in the organic or inorganic chemical industry, analysis of fuels for vehicles directly from the tanks or from feeding pipelines and so on).

For the above described purpose, sampling devices are generally employed that substantially consist of common syringes for medical use, which are mounted (as in the case of amniocentesis, for example) on an already fitted needle or in any case are moved close to the sampling area.

While the just described known art is rather widely spread, it has some drawbacks.

First, execution of known methods for sampling of fluids and/or materials (of organic or non-organic nature) is highly exposed to risks of, contamination of the taken samples, with all negative consequences resulting therefrom.

In addition, referring in particular to organic fluids and/or materials containing stem cells, it is to be pointed out that implementation of a method enabling an efficient and accurate collection and preservation of same is hitherto unknown, neither known is a method capable of ensuring the necessary hermetic sealing and sterility conditions (or in any case the conditions of maximum separation from undesirable environmental agents of macro- or microscopic nature) for putting into practice the “working” and manipulation techniques that can (or could in the future) be applied to the stem cells themselves.

Furthermore, with use of common syringes the collected liquid is required to be poured from the true syringe into a suitable container for storage and/or subsequent analysis; however this intermediate transfer can expose the collected sample to an undesirable contact with the surrounding environment that can lead to contamination and/or mistakes in the analysis.

This drawback is particularly serious in the clinical field, since the possibility of breaking isolation between the fluid collection volume and the surrounding environment can cause entry of pathogenic agents into the sampled fluid, but above all into the patient's body; in the case of prenatal tests, the consequences can be doubly adverse, since obviously possible pathologies can arise both for the pregnant woman and the unborn foetus.

At the same time, it is to be noted that the syringes of known type are not able to preserve the taken fluid sample for an unlimited period of time, due to their intrinsic structure and imperfect hermetic sealing; this makes management and preservation of the taken samples more complicated and in the end increases the analysis costs.

On the other hand, should long-term preservation of the taken fluid be necessary, and in particular should addition of suitable preserving substances to the fluid be required, once more the syringes of the conventional type are not able to carry out this operation without exposing the taken sample to the external environment (which involves exposing the fluid to contamination risks).

In the light of the above, the present invention aims at conceiving a collection and preservation device that is able to obviate the set out drawbacks or in any case to overcome the operating limits of the syringes of the traditional type.

It will be recognised that within the scope of the present invention, the collection and preservation device can be used in the most different operating fields, such as the clinical field for example, but also by way of non-limiting example, in the analysis field and the sampling field concerning non-organic fluid materials.

In particular, the present invention aims at conceiving a collection and preservation device that is able to ensure a full isolation without interruption between the taken sample of fluid and/or material and the external/foreign environment, both at the moment of the true sampling and during the subsequent periods of storage and preservation.

Referring in particular to possible use of this device in the clinical/medical field, an inherent aim of the present invention is also to ensure not only perfect sterility of the taken sample of fluid and/or material, but also perfect sterility of the patent's area where the sampling operations occur.

The present invention also aims at conceiving a collection and preservation device that can be used in the already known analytical sampling methods (both in the clinical/medical field and in other sectors), without said methods being drastically changed from the point of view of their operating sequences.

It is also an aim of the present invention to provide a collection and preservation device having high structural efficiency, high precision in terms of volume of the collected fluid and/or material, low production costs and great ease in use.

From the point of view of the method, the present invention aims at conceiving a process enabling well precise volumes of fluid and/or material to be sampled in a fully safe manner (as regards external contamination) and more quickly than with traditional methods.

Still more generally, the present invention aims at conceiving a method capable of sampling and preserving organic fluids (such as the amniotic fluid for example, and/or the material obtained when a CVS procedure is executed) containing stem cells, so that it is possible to work on the stem cells therein contained and maintained in a preservation state, even after an indefinite period of storage.

Still as regards the method, the present invention wishes to make available a process enabling possible admixing of preserving substances to the sampled fluid and/or material, in a short period of time and with the greatest reliability and accuracy (obviously while fully observing the requirement of isolating them from the external environment also during the step of adding the preservative).

The foregoing and further aims are achieved by a method of collection and preservation of fluids and/or materials, in particular organic fluids and/or materials containing stem cells, as well as by a device employable in such a method, in accordance with the present invention, having the features shown in the appended claims and hereinafter illustrated in an embodiment thereof given by way of non-limiting example, as well as in the accompanying drawings, in which:

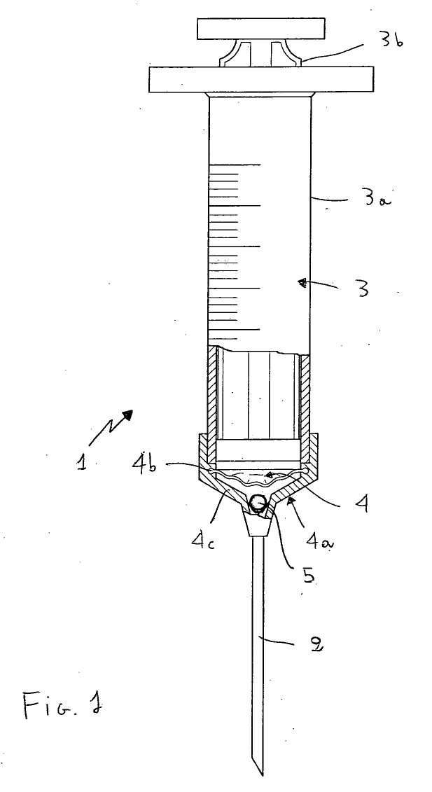

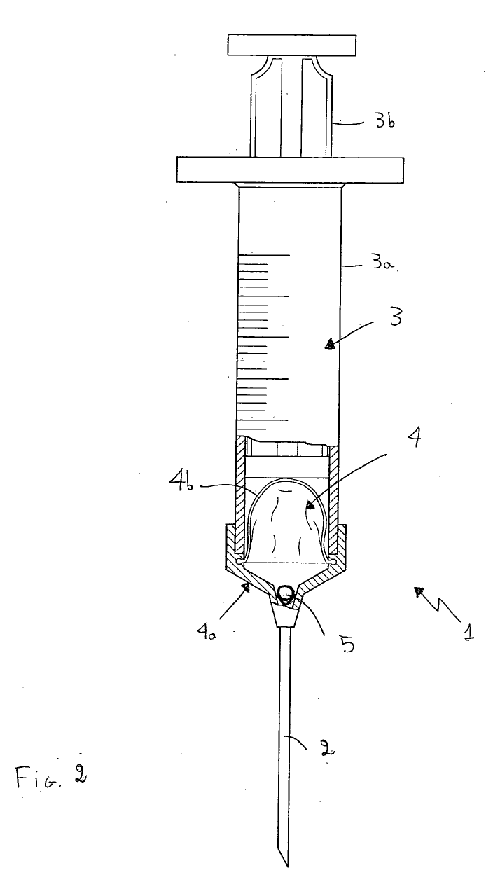

FIGS. 1 and 2 are diagrammatic views of a first embodiment of a device in accordance with the invention, in two different operating configurations; and

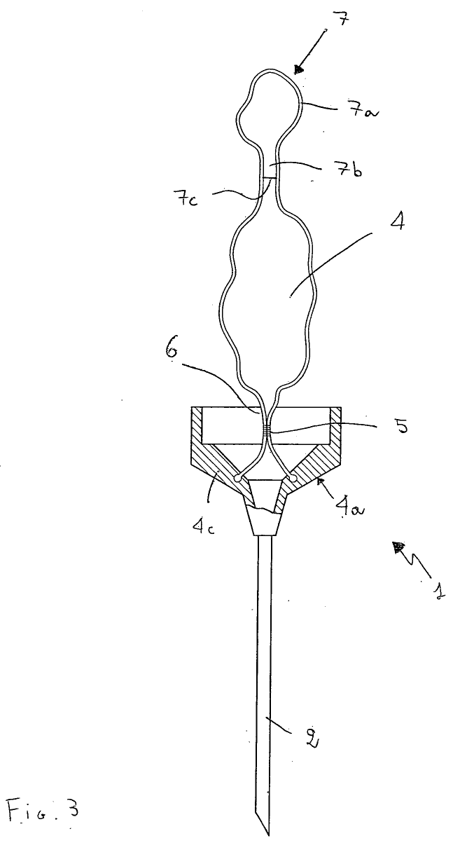

FIG. 3 is a diagrammatic view of a second embodiment of the device in accordance with the invention.

With reference to the drawings, the collection and preservation device in accordance with the invention is generally identified with reference numeral 1 and substantially comprises a series of structural features that will be explained in more detail in the following.

From the operating point of view, the present invention therefore contemplates implementation of a method of collection and preservation of a fluid and/or a material (that can be of organic nature, as in the embodiment of the method described later on, and that preferably will contain a certain amount of stem cells), which mainly (but not exclusively) comprises the following steps:
first of all, a sampling volume is determined, that preferably will be closed and/or separated from the surrounding environment (such as a pregnant woman's placenta, or the inside of a bottle of wine under maturation and so on);
once determined the sampling volume on which to operate, a predetermined amount of fluid and/or material is taken from this sampling volume; and
this predetermined amount of fluid and/or material is confined into a collection volume.

Advantageously, the fluid and/or material sampling and confining are carried out simultaneously, and in addition these two operating steps are contemporaneous with a step of maintaining the hydraulic and/or pneumatic and/or microbiological isolation between the sampling volume and the collection volume.

At this point, it will be appreciated that a difference exists between the method of the invention and the method of the known art; in fact, while in known methods there is always a moment at which the sampled fluid and/or material actually comes into contact with the environment external to the collection volume (let us think of the moment of change of needle in the amniocentesis procedures carried out in a traditional manner or in any case the moment at which the sampling probe is extracted from the collection volume in other fields), according to the present process not only a perfect separation is ensured at any time between the sampled fluid and/or material and the fluid and/or material still remaining in the sampling volume, but at the same time a permanent and continuous separation is ensured over time between the external environment and the amount of sampled fluid and/or material, both during the true sampling and during the subsequent operating steps involving the sampled fluid and/or material.

The method according to the invention is particularly appreciable in the clinical field, where for instance it is possible to operate on the amniotic liquid or also, if necessary, on an organic sample comprising a predetermined amount of chorionic villi; this method can further be used both in traditional amniocentesis and/or CVS operations and in a more complex procedure for extraction and preservation of the amniotic liquid (that in turn is correlated with a preservation process and possible subsequent re-use of the stem cells contained inside the amniotic liquid and/or the chorionic villi).

In the last-mentioned particular application, determination of the sampling volume comprises a sub-step of controlling the foetus' position and/or the placenta's position; in addition, still to the aims of a correct execution of the process (and for reasons connected with control of the health both of the pregnant woman and the foetus), a step is provided for control of a predetermined number of physico-biological parameters of the foetus and/or the pregnant woman.

In more detail in terms of operation, a sampling portion 2 (described in more detail in the following, as well as all the other structural features of the device being the object of the present invention) is introduced which typically comprises at least one needle: this sampling portion is admitted through the mother's abdominal wall and meanwhile a continuous ultrasonographic (echographic) control is carried out in order to increase accuracy of this operation.

The true sampling step starts by (mechanically and/or hydraulically) connecting the sampling portion 2 to a sucking portion 3 (that, depending on particular requirements, can be a traditional cylinder of a piston syringe or any other machine enabling a suction effect to be created on the sampling volume) and/or to a collection portion 4 interposed between the sampling portion 2 and the sucking portion 3.

It is to be noted to the aims of the present invention that by “collection portion 4” it is intended that room permanently isolated and hermetically sealed relative to the external environment inside which the fluid and/or material taken from the sampling volume is collected and confined, while by “sucking portion 3” it is intended that part of device 1 supplying the sucking force required for filling the collection portion 4.

After connecting the above mentioned two portions of the device, a predetermined amount of fluid and/or material is sucked into the sucking portion 3 (and/or into said collection portion 4); within the scope of the present invention, this predetermined amount of fluid and/or material is determined in a precise manner according to the specific requirements and for instance it can be equal to about 3 cc (or more generally, it can be an amount included between 1.5 cc and 10 cc) in the case of an amniocentesis or CVS procedure (or more generally, if a sample of amniotic liquid is wished to be preserved for the most varied purposes).

When the desired amount of fluid and/or material has been sampled, the amount of fluid and/or material sucked into the collection portion 4 is hermetically isolated; this hermetic isolation is carried out, according to a particularly appreciable feature of the present invention, by (automatic and instantaneous) closure of suitable hydraulic nonreturn means.

Finally, after defining a well precise amount of sampled fluid and/or material and having at the same time maintained a full isolation from the external environment during the whole sampling sub-step, the procedure goes on by separating the sampling portion 2 from the sucking portion 3 and/or the collection portion 4; if subsequent sampling operations are wished to be carried out (as in the case of traditional amniocentesis for example, where the first small sampling is followed by a much more solid sampling of amniotic liquid, in order to carry out the suitable analyses thereon), the sampling portion 2 can be left in fluid communication with the sampling volume and can therefore receive a new sucking portion 3 in engagement (of a device similar to or different from the one shown in the present invention).

As regards operation, it is therefore possible to contemplate a step of taking an additional sample of fluid and/or material that will be carried out subsequently to the sub-step of separating the sampling portion 2 from the sucking portion 3 (and/or from the collection portion 4) and that can in turn comprise a sub-step of mechanically and/or hydraulically connecting the sampling portion 2 (that is still fitted into the sampling volume) to a new sucking portion 3 and/or a new collection portion 4 interposed between the “old” sampling portion 2 and the new sucking portion 3 itself.

To the aims of the present invention, it is important to emphasise that maintenance of the hermetic sealing and/or isolation between the sampling volume and the external environment is also ensured at the moment of separation of the sucking portion 3 (or the collection portion 4) from the sampling portion 2; this takes place due to the presence of suitable isolation means automatically and instantaneously starting operation at the separation instant (this means can be, depending on particular requirements, nonreturn valves or previously calculated narrowing passages of the needle or others).

The present invention can also ensure hermetic separation between the sampling volume and the sampling portion 2, both during transfer of the fluid and/or material and even when the sampling portion 2 itself is separated from the sampling volume; in particular, the presence of suitable sealing means for the sampling volume can be provided, which means is operatively activated on said volume when the sampling portion 2 is extracted from the sampling volume and/or moved away therefrom.

This sealing means can be made in different ways; for instance, for clinical applications or applications to be experienced on patients' bodies, a sheath of a material having suitable sealing properties and that can be submitted to spontaneous contraction can be provided; this sheath externally covers the sampling portion 2 and remains inserted through the wall defining the sampling volume when extraction of the sampling portion 2 takes place (as already said, this sampling portion can consist of a hypodermic needle or the like).

By suitably selecting sizes and materials, this sheath (that can also be bio-degradable and bio-compatible) will form a plug when the needle is extracted, thus immediately and stably sealing the hole formed in the patient's body until it will be gradually absorbed by the body itself during the healing process.

Advantageously, the sealing means mentioned above can be structurally and/or operatively connected to any type of collection and preservation device, and be also used in any operating method requiring the presence of same.

Optionally, the method hereabove described can be integrated with a step of storing and preserving the fluid and/or material; this operating step can conveniently comprise a sub-step of spraying and/or mixing the fluid and/or material with at least one preserving agent (for example a dimethyl sulfoxide-based preservative, or DMSO as commonly termed in the chemical field).

In more detail, it is possible to see that the spraying/mixing sub-step is carried out through selective opening of a hydraulic locking element 5 interposed between a spraying element 7 and the collection portion 4 and/or between the sucking portion 3 and the collection portion 4; in this manner it is possible to select the suitable moment for mixing the preservative with the fluid and/or material (this is in particular useful when DMSO is used because it can be added to organic fluids only under determined temperature conditions).

In accordance with the present invention, the spraying/mixing step too takes place while ensuring the full separation and hermetic sealing of the collected fluid and/or material relative to the surrounding environment; this special quality is ensured by a suitable construction of device 1 which is able to selectively bring the sampled fluid and/or material into communication with the preserving agent without any need for the latter to be taken from the outside.

Conveniently, the step of preserving the sampled fluid and/or material further comprises a sub-step of cooling the fluid and/or material under a predetermined preservation temperature; this cooling sub-step can precede the sub-step of spraying and/or mixing the fluid and/or material with at least one preserving agent.

After the desired amount of fluid and/or material has been taken, and after possibly doing the necessary to enable preservation of same for an indefinite period of time, it is also possible to transfer the fluid and/or material into suitable storage means.

This step of transferring the fluid and/or material into storage means can be conveniently optimised from the logistic point of view by allocating a series of data at least concerning positioning (but also, depending on specific requirements, data relating to other parameters, such as sampling data, environmental conditions at the sampling moment, operator who has carried out sampling, and so on) to each fluid and/or material sample; subsequently, these data can be stored into suitable storage systems.

As regards execution, the present method can advantageously apply for collection and preservation (as well as for a possible and subsequent new processing or culturing) of organic fluids and/or materials containing stem cells (such as the amniotic liquid and/or the chorionic villi that can be taken from a pregnant woman, for example).

It is to be noted in this connection that stem cells taken from the amniotic liquid can be used as a cellular therapy source for treatment of pathologies in humans: in order to ensure use of these cells it is of the greatest importance to ensure sterility of the cellular product with which the patient will be re-inoculated.

Operatively, sterility of the amniotic liquid sample (or the sample of chorionic villi) during all the manipulation steps following true sampling, or possibly also the previously described preservation/storage step, is ensured by a suitable laboratory instrument known in the technical field with the name of “isolator”.

It is to be noted at this point that use of the isolator constitutes an important novelty relative to the methodologies presently applied for manipulating stem cells: actually, operators presently acting on stem cells move (and manipulate) the biological material inside sterile rooms and therefore, although they have all possible protections (masks, gloves and others) at their disposal, they yet represent a contamination source for the organic fluid or material under processing, since they transfer (at least through breathing) an important charge of bacteria or in any case of polluting agents (powders and dust, residues resulting from exfoliation of the skin and so on) into the same environment where the stem cells are.

On the contrary, use of the isolator enables full separation between the operator (and above all any environmental alteration/pollution source connected with the operator's physical presence) and the fluid and/or material containing the stem cells, or even the stem cells already separated from the fluid/material together with which they have been taken from a given patient.

From a structural point of view, the isolator employable in the present method mainly comprises a work chamber which practically is a steel box completely isolated from the outside, provided with a filtering system having filters of the “Hepa” type and where access for the operator takes place through use of suitable gloves jutting out inside the box and sealingly connected with one of the box walls: conveniently, this box is pressurised to a greater pressure than the inlet chamber.

Also present is an inlet chamber (also termed “pass-box”) for introduction of the sample and the biological material to be processed, which chamber is provided with interlocking doors that do not allow direct communication between the work chamber and the surrounding environment.

Likewise, an outlet chamber is present which has a sterile sample-collecting bag: this outlet chamber is brought into communication with the work chamber by interlocking doors.

Finally, a sterilisation system is present for processing the biological sample and sterilising the outer surface of the biological-sample container; the sterilisation process takes place by use of hydrogen peroxide (H₂O₂) that is introduced into the isolator before each work step and between processing of two different biological samples in order to ensure absolute sterility during the manipulation step; during this step the “particle count” and/or “microbiological count” parameters will be continuously analysed.

On the contrary, as regards freezing of the stem cells, the following is done.

From about 20 ml of amniotic liquid obtained through amniocentesis (or from a corresponding amount of chorionic villi, obtained through CVS—Chorionic Villus Sampling), an aliquot part of 2-2.5 ml will be taken, then the samples contained in 15 ml test tubes with conical bottom and screw plug are centrifuged at 2000 rpm for 10 minutes.

After centrifugation, the test tube is inserted into the isolator and without disturbing the so-called “cellular pellet”, the so-called “supernatant” is taken and preserved for preparing the freezing solution for the sample with final 10% DMSO.

This solution is cooled using a suitable cooling apparatus positioned inside the isolator and 1 ml thereof is used to suspend the amniocyte pellet again, said pellet being then inserted into a suitable test tube for freezing (which instead has been caused to come out of the isolator's outlet chamber and then frozen by a programmable freezer).

At the end of the freezing step, the sample is preserved in suitable storage containers containing liquid nitrogen.

Generally freezing is carried out on non-hematic samples and on samples non containing meconium that have been taken 24-48 hours earlier.

Defrosting of the stem cells is carried out by taking the sample out of the liquid nitrogen, positioning it in ice and bringing it into a 37° C. thermostat in the shortest period of time.

After about 3 minutes, the defrosted sample is transferred into the isolator and then drop-wise transferred into a 15 ml test tube with conical bottom and screw plug (this test tube contains about 9 ml of washing medium).

At the end of the addition, the test tube is caused to come out of the isolator's outlet chamber and is subsequently centrifuged at 1500 rpm for 10 minutes.

After centrifugation, the test tube is inserted into the isolator and without disturbing the cellular pellet, the supernatant is taken; at this point, the cell-containing pellet is suspended again with a suitable cell-growth substance (termed “medium”) in an amount of about 2 ml; the cellular suspension will be transferred into a suitable flask (in jargon termed “T25”) for expansion.

It is to be noted that in accordance with the present invention, the method of collection and preservation of organic fluids and/or materials containing stem cells (such as the amniotic liquid and/or chorionic villi that can be taken from a pregnant woman) can therefore comprise a criogenic preservation step and possibly a subsequent unstoring step that in turn comprises at least a defrosting sub-step.

More generally, it is also to be noted that the collection and preservation method applicable to organic fluids and/or materials containing stem cells can advantageous comprise a sampling step (carried out on a suitable “collection volume”) that is executed while a perfect microbiological, atmospheric and physical isolation is continuously maintained between the sampling volume, the sample of collected fluid and/or material and the collection portion wherein the fluid/material is confined.

As regards the above mentioned isolator, it is to be noted that within the scope of the present invention the structure of the latter has been suitably conceived for maximising the efficiency of the work method.

First of all, it will be appreciated that the size of the isolator's work chamber (which can be considered by way of example as a cubic volume having sides of about 80 cm) has been suitably studied so as to reduce the sizes of the inner surfaces and the volume: this geometric effect greatly reduces the time required for decontamination of the work chamber and also reduces consumption of sterilising agents (such as hydrogen peroxide) required for decontamination.

The isolator also offers the possibility of pre-cooling the freezing solution by using a thermo-block (made of steel) housed inside the work chamber: this positioning of the thermo-block ensures maximum sterility for the biological sample and the work area, unlike known systems exploiting a more traditional cooling by ice (which is not sterile and cannot be sterilised) contained in a container that in turn is not sterile.

The structural features of the isolator in this manner offer the possibility of keeping the biological sample inside the work chamber at a controlled temperature (i.e. temperature-regulating means is present in the work chamber, so that suitable comfort conditions can be achieved both for the product and the operator).

At the same time the isolator is provided with sealed and/or hermetic and/or sterile packaging means located at the outlet chamber: this sealed and/or hermetic and/or sterile packaging means offers the possibility of packaging the product coming out of the isolator in an aseptic manner, by a suitable sterile “rolled bag” (which in turn is useful for the purpose of carrying the sample to the cryogenic freezing and/or cryogenic preservation point).

In addition, due to the presence of the sterile “rolled bag” or equivalent means, possible working waste can also be eliminated without contaminating any part of the isolator (and therefore avoiding further sterilisation cycles being carried out).

The present isolator further has suitable filters (made of Gore-tex for example) that are operatively active on a line for admission of sterilising agents, and preferably a line for admission of hydrogen peroxide; in this manner a further lowering of the decontamination time is obtained.

In order to reach an ergonomic improvement for the operator, the isolator also has suitable positioning means for a biological sample (or in other words, for the sampled fluid and/or material), as well as for the material required for processing: conveniently, this means can consist of a steel rack having the same surface finish degree as the isolator's walls.

The isolator can also be provided with self-governing movement means (a train of pivoting wheels or the like, for example) that allow displacement of same inside the room: practically, the self-governing-movement means allows the isolator to be shifted to the desired points, thus facilitating cleaning of the room and maintenance of the isolator itself, for example (or in any case offering the possibility of shifting the isolator to points in the room or laboratory that are more advantageous from an operating point of view).

As already said, the isolator also comprises sterilisation means acting at least on the work chamber and/or the inlet chamber and/or the outlet chamber: this means can conveniently atomise one or more sterilising agents so as to decontaminate every point of the isolator itself.

For completion of the different operating aspects of the isolator, suitable sensor means is then present which acts at least in the work chamber (but, if necessary, also in the inlet chamber and/or the outlet chamber), said sensor means being able to measure:
a microbiological sampling (typically, by an air intake positioned close to the work region, so as to have a truer and safer view of the working neighbourhood;
an air speed within the work chamber (these sensors can be possibly coupled with filtering means, comprising one or more filters of the “hepa” type for example, and/or with venting means acting on the work chamber and/or the inlet chamber and/or the outlet chamber);
a given number of particle values of the air during the whole process;
a possible residual amount of sterilising agents still in the work chamber after a sterilisation process.

Depending on the different occasions, the above described isolator can also be used separated from the method being the object of the present invention, i.e. in other industrial and/or laboratory processing methods on several different types of materials and/or (biological and non-biological) samples.

As already said during the present specification, it is a further object of the invention to provide a device for collection and preservation of fluid material, which comprises:
a sampling portion 2 adapted to be brought into fluid communication with a sampling volume;
a sucking portion 3 operatively connected to the sampling portion 2 and adapted to be operatively activated for recalling or sucking a predetermined amount of fluid from the sampling volume; and
a collection portion 4 interposed between the sampling portion 2 and said sucking portion 3 (and that, as already explained, receives the sampled fluid).

Advantageously, the collection portion 4 comprises an expandable element 4a defining a collection volume, and at the same time this collection volume is brought into fluid communication with the sampling volume but is hermetically sealed relative to the surrounding atmosphere.

The expandable element 4a can be configured in a reversible manner between a rest condition defining a minimum or zero collection volume and a maximum-filling condition at which on the contrary it defines a maximum collection volume.

The above mentioned expandable element 4a is therefore able to change its conformation in space, but according to the present invention it is to be considered as substantially undeformable (or at all events not further deformable) at its configuration defining the maximum collection volume.

As shown in the accompanying figures, the expandable element 4a can consist of an elastic membrane suitably mounted inside a head portion of a syringe (that in turn can be removable relative to the sucking body of the syringe itself that in turn holds a piston or equivalent sucking means).

Conveniently, the just described membrane can be replaced or integrated with an element operating in the same manner, such as a movable wall suitably connected in a sealing manner with a corresponding fixed wall.

Alternatively, and again referring to the accompanying drawings, the expandable element 4a can consist of a bag connected to the sampling portion: this bag is progressively filled and inflated while fluid sampling is being carried out.

By a suitable selection of sizes and/or materials, it is possible to accurately establish the maximum volume that this expandable element 4a can define: for instance, when amniotic liquid or chorionic villi are to be sampled, this maximum collection volume can be fixed to 3 cc.

Still with reference to the drawings (and in particular with reference to FIG. 1), the sucking portion 3 comprises a holding body 3a of substantially cylindrical shape and a slider 3b the shape of which matches that of the holding body 3a; this slider 3b is slidably movable within the holding body 3a and the expandable element 4a too is contained (at least partly) in the holding body 3a itself; the expandable element 4a becomes deformed at least at a part thereof, by effect of a sucking action that can be exerted by said slider 3b.

In more detail with reference to FIG. 1, it is possible to see that the expandable element 4a comprises a hermetic base 4c susceptible of being interposed (preferably in a removable manner) between the sampling portion 2 and the sucking portion 3 (preferably close to one end of the sucking portion 3 itself), and a diaphragm 4b having a peripheral edge hermetically connected to an inner wall of the collection and preservation device 1.

Typically, the inner wall of the collection and preservation device 1 can be an inner wall of the sucking portion 3 and more particularly an inner wall of the hermetic base 4c.

With reference to FIG. 2, it is possible to see that an inflatable bag in the form of a balloon can be instead inserted into the hermetic base 4c, said bag being possibly provided with a (preferably flexible) hose connected to the sampling portion 2.

Advantageously, at least one hydraulic locking device 5 can be present which is interposed between the sampling portion 2 and the expandable element 4a, so as to lock the flow of the collected fluid in an arbitrary manner and at the same time in order to lock possible refluxes in the opposite way of the return fluid towards the sampling volume.

Depending on specific requirements, this hydraulic locking element 5 can be a nonreturn valve (that typically can be inserted in the embodiment in FIG. 1, close to the base of the sampling portion 2 or in an interfacing portion of the hermetic base 4c) or, as shown in the alternative embodiment in FIG. 2, can consist of a narrowing passage or neck formed in the sampling portion 2 and/or the hermetic base 4c and/or the diaphragm 4b.

With reference to the last-mentioned embodiment, it is possible to see that in accordance with the invention the narrowing passage can be formed in different ways, such as by striction with suitable mechanical means (clips, tying elements and others) or by heat sealing, ultrasonic wave welding and the like.

Conveniently, also present may be at least one connecting element 6 interposed between the sampling portion 2 and the expandable element 4a: this connecting element can consist of a deformable tubular duct.

According to a further feature typical of the present invention a hydraulic locking element 5 may be also present, said element being placed within the connecting element 6 (and as already said it can consist of a narrowing passage preferably obtained by heat sealing or ultrasonic wave welding).

In order to be able to implement the (optional) method step involving spraying with a preservative, device 1 can comprise a spraying element 7 containing a predetermined amount of additive fluid; the spraying element 7 is connected at least to the expandable element 4a and can be configured in a reversible manner between a hydraulic isolation condition at which no exchange between said additive fluid and the collection volume occurs and an inflow condition at which flowing of the additive fluid into the collection volume is allowed.

From a structural point of view, the spraying element 7 (that is visible in the embodiment in FIG. 2 although it is optional both for this embodiment and the embodiment in FIG. 1) comprises a deformable bag 7a containing the additive fluid (a dimethyl sulfoxide-based preservative—DMSO—for example) and a feeding duct 7b interposed between the deformable bag 7a and the collection volume.

Conveniently, in order to be able to state the moment for carrying out spraying of the additive fluid in the most appropriate manner, hydraulic separation means 7c is placed in the feeding duct 7b and said means can be configured between a locked condition at which it does not allow flowing of the additive fluid towards the collection volume and an unlocked condition at which it allows flowing of the additive fluid towards the collection volume.

From a structural point of view, said hydraulic separation means 7c can be based on a predetermined number of frangible walls breaking of which is previously calculated but if necessary said means can be replaced by equivalent elements (valves or others).

The invention enables achievement of important advantages.

First of all, it will be recognised that the particular construction architecture of the device herein illustrated (and subsequently claimed) enables collection of a fluid in a closed volume (the capacity of which is stated in the most accurate manner) in a very quick manner, which fluid is maintained constantly isolated relative to any type of surrounding environment or “external” agent.

It will be also appreciated that the present device simultaneously carries out collection and storage of the fluid sample, thus reducing the operating time and making the fluid sample immediately available, without intermediate pouring operations being required.

In addition, possible use of the present device in the medical/clinical field allows higher hygiene and sterilisation parameters to be maintained also as regards the patient's body that is not exposed to several perforations in succession with similar needles or instruments.

Secondly, the present device can be made in such a manner as to enable possible preserving agents to be admixed while, on the one hand, ensuring the maximum measuring accuracy and full control of the mixing moment and also ensuring, on the other hand, that mixing between sampled fluid and preservative takes place without possible contacts with the surrounding environment.

In terms of operation, the present invention therefore enables accomplishment of a quicker collection and preservation method as compared with traditional methods in use; this method also allows two operations that are generally carried out at different times (and therefore are time-consuming) to be integrated into a single operating step.

It will be appreciated that this method is in any case compatible with the already known operating methodologies and that a different degree of preparation by the staff putting it into practice is not required.

Finally, the present invention enables low production and sale costs to be achieved both in terms of manufacture of the collection and preservation device and in terms of cheap management of the sampling/storage/analysis works that are necessary in a great number of technological fields.

1-36. (canceled)

37. A method of collection and preservation of a fluid and/or a material, said fluid and/or material being of organic nature and containing stem cells, comprising the following steps:
determining a sampling volume that is closed and/or separated from the external environment;
taking a predetermined amount of fluid from said sampling volume; and confining said predetermined amount of fluid into a collection volume, said step of taking a predetermined amount of fluid and said step of confining said predetermined amount of fluid being simultaneous with each other and being also contemporaneous with a step of maintaining the hydraulic and/or pneumatic and/or microbiological isolation between the sampling volume and the collection volume; and
storing and preserving the fluid and/or material, said step of storing and preserving the fluid and/or material comprising a sub-step of spraying and/or mixing the fluid and/or material with at least one preserving agent, said preserving agent comprising a predetermined amount of dimethyl sulfoxide.

38. A method as claimed in claim 37, wherein said fluid and/or material is the amniotic liquid and/or comprises a predetermined amount of chorionic villi, said step of determining a sampling volume further comprising a sub-step of controlling a position of at least one foetus and/or a position of a placenta and/or of a predetermined number of physico-biological parameters of said foetus.

39. A method as claimed in claim 38, wherein the step of determining a sampling volume further comprises a sub-step of introducing a sampling portion (2) comprising at least one needle through the mother's abdominal wall, said sub-step of introducing said sampling portion (2) being carried out under a continuous ultrasonographic control.

40. A method as claimed in claim 37, wherein the step of taking a predetermined amount of fluid from the sampling volume comprises the following sub-steps:
mechanically and/or hydraulically connecting the sampling portion (2) to a sucking portion (3) and/or to a collection portion (4) interposed between the sampling portion (2) and the sucking portion (3);
sucking a predetermined amount of fluid into the sucking portion (3) and/or into said collection portion (4), said predetermined amount of fluid being included between 1 and 10 cc;
hermetically isolating the sucked amount of fluid and/or material in the collection portion (4) through closure of hydraulic non-return means; and
separating the sampling portion (2) from the sucking portion (3) and/or from the collection portion (4), the sampling portion (2) remaining in fluid communication with the sampling volume.

41. A method as claimed in claim 40, wherein said predetermined amount of fluid is equal to 3 cc.

42. A method as claimed in claim 37, wherein also present is a step of taking an additional sample of fluid and/or material, said step taking place after the sub-step of separating the sampling portion (2) from the sucking portion (3) and/or from the collection portion (4), and comprising a sub-step of mechanically and/or hydraulically connecting the sampling portion (2) to a new sucking portion (3) and/or to a new collection portion (4) interposed between the sampling portion (2) and the new sucking portion (3).

43. A method as claimed in claim 37, wherein said sub-step of spraying and/or mixing the fluid and/or material with a preserving agent takes place through selective opening of a hydraulic locking element (5) interposed between a spraying element (7) and the collection portion (4) and/or between the sucking portion (3) and the collection portion (4).

44. A method as claimed in claim 37, wherein the step of preserving the sampled fluid and/or material further comprises a sub-step of cooling the fluid and/or material under a predetermined preservation temperature, said sub-step of cooling the fluid and/or material taking place before the sub-step of spraying and/or mixing the fluid and/or material with at least one preserving agent.

45. A method as claimed in claim 37, wherein also present is a step of transferring the fluid and/or material into storage means, said step of transferring the fluid and/or material into storage means comprising a sub-step of allocating data relating to at least positioning to a predetermined sample of fluid and/or material, and a subsequent sub-step of storing said data relating to at least positioning.

46. A method of collection and preservation of organic fluids and/or materials containing stem cells, said fluids and/or materials comprising an amniotic liquid and/or chorionic wherein at least one step according to claim 37 is present.

47. A method as claimed in claim 46, wherein also present is a step of manipulating the sampled fluid and/or material, said step of manipulating the fluid and/or material being carried out under sterility conditions and being performed by an isolator apparatus.

48. A method as claimed in claim 47, wherein said isolator apparatus comprises:
a work chamber isolated from the external environment and provided with a filtering system, an operator being able to accede to said work chamber by use of suitable gloves jutting out at the inside of the box and sealingly connected with one of the work chamber walls, said work chamber being pressurised to a greater pressure than the inlet chamber;
an inlet chamber for introduction of a sample and/or a biological material to be processed, said inlet chamber being connected to the work chamber and being provided with interlocking doors that do not allow direct communication between the work chamber and the external environment;
an outlet chamber connected with the work chamber and having a sterile sample-collecting bag, said outlet chamber being brought into communication with the work chamber by means of interlocking doors; and
a sterilisation system for processing the biological sample and sterilising an outer surface of a container of the biological sample itself, the sterilisation process taking place by use of hydrogen peroxide introduced into the isolator before each work step and between the operations for processing two different biological samples.

49. A method as claimed in claim 46, wherein a step is present for continuous control of particle-count and/or microbiological-count parameters.

50. A method as claimed in claim 46, wherein also present is a step of freezing the stem cells.

51. A method as claimed in claim 50, wherein said step of freezing the stem cells comprises the following sub-steps:
taking an amount of an amniotic liquid and/or chorionic villi, obtained through CVS (Chorionic Villus Sampling), said amount being equal to 2-2.5 ml; and
admitting said amount into a container, said container being a 15 ml test tube with a conical bottom and a screw plug; and
centrifuging said amount of an amniotic liquid and/or chorionic villi, said centrifuging sub-step being carried out at 2000 rpm for 10 minutes.

52. A method as claimed in claim 51, wherein a step of inserting said test tube into the isolator is present and wherein further present is a step of taking a supernatant from said test tube.

53. A method as claimed in claim 46, wherein also present is a step of freezing the sample added with 10% DMSO (dimethyl sulfoxide), said freezing step comprising the following sub-steps:
cooling the sample, using a suitable freezing apparatus positioned within the isolator;
suspending the amniocyte pellet again, said amniocyte pellet being subsequently inserted into a test tube for freezing;
extracting said test tube for freezing from the isolator through said outlet chamber of the isolator; and
freezing said test tube for freezing by means of a programmable freezer, said step of freezing the sample being carried out on non-hematic samples and on samples non containing meconium that have been taken 24-48 hours earlier.

54. A method as claimed in claim 53, wherein also present is a step of preserving the sample in suitable storage containers containing liquid nitrogen.

55. A method as claimed in claim 46, wherein also present is a step of defrosting the stem cells.

56. A method as claimed in claim 55, wherein said defrosting step comprises the following sub-steps:
taking the sample from liquid nitrogen;
positioning the sample in ice; and
bringing the sample into a thermostat at 37° C.

57. A method as claimed in claim 55, wherein a step of transferring the sample into the isolator is provided after said defrosting step, and wherein a step of transferring the sample drop-wise into a test tube is present, said test tube having a 15 ml capacity, conical bottom and screw plug.

58. A method as claimed in claim 57, wherein said test tube contains about 9 ml of a washing medium.

59. A method as claimed in claim 57, wherein also present is a step of centrifuging said test tube, said centrifuging step being carried out after a sub-step of extracting the test tube from the isolator and being carried out at 1500 rpm for 10 minutes.

60. A method as claimed in claim 59, wherein also present are the following steps:
subsequently to the step of centrifuging a test tube, inserting said test tube into the isolator;
taking a supernatant from said test tube;
re-suspending a cell pellet contained in said test tube with a suitable growth substance in an amount included between 1 ml and 4 ml; and
transferring said cell pellet and said growth substance into a flask of the “T25” type.

61. A method of collection and preservation of organic fluids and/or materials as claimed in claim 37, wherein said fluid and/or material contains stem cells, and wherein also present are the following steps:
taking or sampling the fluid and/or material while continuously maintaining a perfect microbiological, atmospheric and physical isolation between the sampling volume, the sample of collected fluid and/or material and the collection portion wherein the fluid/material is confined;
preserving said fluid and/or material in a cryogenic manner; and
subsequently to said step of preserving the fluid and/or material in a cryogenic manner, unstoring said fluid and/or material, said step of unstoring the fluid and/or material comprising a defrosting sub-step.

62. A device for collection and preservation of fluid materials, comprising:
a sampling portion (2) adapted to be brought into fluid communication with a sampling volume;
a sucking portion (3) operatively connected to said sampling volume (2) to suck a predetermined amount of fluid and/or material from the sampling volume; and
a collection portion (4) interposed between the sampling portion (2) and said sucking portion (3), said collection portion (4) comprising an expandable element (4a) defining a collection volume, said collection volume being brought into fluid communication with the sampling volume and being hermetically sealed relative to the external environment; and
a spraying element (7) containing a predetermined amount of additive fluid, said spraying element (7) being connected at least to the expandable element (4a) and being configured in a reversible manner between a hydraulic-isolation condition at which no exchange between said additive fluid and the collection volume takes place and an inflow condition at which flowing of the additive fluid into the collection volume is allowed.

63. A device as claimed in claim 62, wherein said expandable element (4a) comprises a membrane that can be configured in a reversible manner between a rest condition defining a minimum or zero collection volume and a maximum-filling condition at which on the contrary it defines a maximum collection volume, said maximum collection volume being equal to 3 cc.

64. A device as claimed in claim 62, wherein the sucking portion (3) comprises a holding body (3a) of cylindrical shape and a slider (3b) the shape of which matches that of said holding body (3a) and which is slidably movable at the inside of the latter, the expandable element (4a) being at least partly contained in said holding body (3a) and being submitted to deformation by effect of a sucking action that can be exerted through said slider (3b).

65. A device as claimed in claim 62, wherein the expandable element (4a) comprises:
a hermetic base (4c) adapted to be interposed between the sampling portion (2) and the sucking portion (3) in the vicinity of one end of the sucking portion (3); and
a diaphragm (4b) having a peripheral edge hermetically connected to an inner wall of the collection and preservation device (1), said inner wall of the collection and preservation device (1) being an inner wall of the sucking portion (3) and of the hermetic base (4c).

66. A device as claimed in claim 62, wherein also present is at least one hydraulic locking element (5) interposed between the sampling portion (2) and the expandable element (4a), said hydraulic locking element being a non-return valve or a narrowing passage formed in the sampling portion (2) and/or the hermetic base (4c) and/or the diaphragm (4b).

67. A device as claimed in claim 62, wherein also present is at least one connecting element (6) interposed between the sampling portion (2) and the expandable element (4a), said connecting element being configured in the form of a deformable tubular duct.

68. A device as claimed in claim 66, wherein said at least one hydraulic locking element (5) is placed in said connecting element (6), said hydraulic locking element being a narrowing passage or neck and being obtained by heat sealing or ultrasonic wave welding.

69. A device as claimed in claim 62, wherein the spraying element (7) comprises:
a deformable bag (7a) containing the additive fluid, said additive fluid being a preservative, said preservative being based on dimethyl sulfoxide;
a feeding duct (7b) interposed between said deformable bag (7a) and the collection volume; and
hydraulic-separation means (7c) placed in said feeding duct (7b) and configured between a locked condition at which said means does not allow flowing of the additive fluid towards the collection volume and an unlocked condition at which it allows flowing of the additive fluid towards the collection volume.

70. A device as claimed in claim 69, wherein said hydraulic-separation means (7c) comprises a predetermined number of frangible walls with previously calculated breaking.
